(19) United States
(12) Patent Application Publication
(10) Pub. No.: US 2004/0064165 A1
(43) Pub. Date: Apr. 1, 2004

(54) Implantable medical device with hardware-based patch

(76) Inventor: David L. Thompson, Andover, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/260,943

(22) Filed: Sep. 30, 2002

(51) Int. Cl. A61N 1/08

(52) U.S. Cl. 607/59

(57) ABSTRACT

An implantable medical device is provided which provides for more flexibility in re-programming of the control software, or program, which is controlling the functionality of an implanted pacemaker. The system comprises an external programmer device, which is capable both of programming the implanted device in a conventional way, and also downloading new control software to the implanted device. The downloaded software is programmed into a PLD integrated into the microprocessor, I/O, memory, and a clock control system via a standard data bus. The PLD may be configured to modify the function of the microprocessor, system I/O, random logic, and/or firmware function. The system enables increased processing capabilities, speed, flexibility, and attenuation of battery current drain.

### FIELD OF THE INVENTION

The present invention relates to programmable implantable medical devices and, more particularly, to systems and methods for downloading a new or modified program into a programmable, customizable section of the implanted medical device.

### BACKGROUND OF THE INVENTION

Implantable medical devices, and in particular stimulus devices such as cardiac pacemakers, cardioverters, defibrillators, drug pumps, and neuro stimulators have for some time been software programmable. By software programmable, it is meant that the implanted device contains a form of microprocessor or microcomputer, and associated memory, the memory containing a control program for controlling prescribed device operations. Such programmability or software control has become necessary with the advent of more sophisticated and complex pacemaker devices, wherein real time operation can be achieved only with microprocessor-based control. For example, with the increased use of DDD pacing, and rate responsive pacing, as well as ongoing collection of events for diagnostic purposes, exclusive hardware control may not be feasible.

The demands for microprocessor control led to the development of pacemakers with platforms, or main building blocks, wherein the pacemaker could be modified by the software that was downloaded into its memory. This technique enables producing different pacemaker types at the factory, or manufacturing site, by the expedient of loading the appropriate control program or programs into non-volatile memory (i.e., ROM) of the pacemaker. The use of microprocessor-based pacemakers also enables a subsequent update of already implanted pacemakers, by downloading new control programs, or software, through the use of commercially available external programmer devices. Such capability for downloading new control program software into an implanted parameter enables building a device platform, which is flexible enough to be software modified in the future to adapt it for different applications and studies. For example, a pacemaker can be upgraded with new diagnostic tools and therapies to study the onset and prevention of atrial tachyarrhythmias. Alternatively, a pacemaker implanted in a patient that is subsequently obsoleted or rendered ineffective by a different heart failure mode would be modified to enable an appropriate new therapy and to carry out new diagnostic data accumulation based on changing patient therapy requirements. See, for example, U.S. Pat. No. 5,360,437 to Thompson incorporated herein by reference in its entirety. Additionally, correction and/or improvements of features or function in an implanted pacemaker are also possible by downloading software “patches”, eliminating the need for explant of the device and subsequent cost and trauma to the patient. As is well known in the art, downloading of new software into an implanted pacemaker, i.e., using an external programmer to transmit a new control program for memory storage in the pacemaker, depends upon access to a programmer. The programmer must be capable of providing the desired software modification(s), and provide conventional programming of the pacemaker, e.g., setting stimulus pulse parameters, rate limits, modes of operation, sensing parameters, etc.

Typical prior art pacemaker systems generally include means for storing control software in read only memory (“ROM”) and random access memory (“RAM”), and an external programmer and a telemetry link for downlinking code to the RAM, which downlinked code is capable of causing the implanted medical device to perform new and/or revised functionality. The firmware, also referred to as control software, is stored in such a manner that the amount of code stored in ROM is maximized and RAM storage is used for diagnostic data, storing pointers to functions, the relative priority and/or execution order of functions, and any code downlinked via the external programmer and telemetry link. When code is downlinked to and stored in RAM, one or more revised and/or new pointers to the code that has been downlinked to RAM are also downlinked and stored in RAM. In this manner, execution of ROM-based tasks can be temporarily suspended by downlinking a pointer to execute a downlinked task in RAM, completing that task, and then returning to the program stored in ROM.

Prior art pacemaker downloadable code programs or patches are described in U.S. Pat. No. 5,843,138 to Evers, et al; U.S. Pat. No. 6,200,265 to Walsh, et al; and U.S. Pat. No. 6,282,450 to Hartlaub, et al; all incorporated herein by reference in their entireties.

While prior art downloadable patches or code have been useful, they have some limitations. For example, typical downloadable patches or code are stored in RAM memory taking up memory that is normally used for diagnostics data storage and thus limiting the diagnostic features and functions of a device with a downloadable patch. Additionally, code run out of RAM memory consumes more battery power than ROM based code or random logic implementations, as the code must be accessed over a system bus thus shortening the useful life of an implanted device. Further, generally only one download patch may be used at a time in an implanted device limiting its flexibility and usefulness. While the prior art has disclosed modifying control programs in implanted pacemakers, using external programmers and telemetric transmission, there remains a need for a system which ensures reliable and low power control program modification, i.e., repairing, upgrading or down-grading, of implanted devices. There remains a need for a system for efficiently implementing the program changes that are made to any implanted medical device. Consequently, there is a need to provide a program modification system for use with an implanted medical device, and particularly, a pacemaker, which efficiently and reliably operates in the implanted device.

### SUMMARY OF THE INVENTION

One aspect of the present invention relates to implantable device system, preferably a cardiac pacemaker system, having two-way telemetric capability between an implanted pacemaker and an external programmer unit. The pacemaker preferably includes memory for storing a control program, which must be non-invasively modified from time to time. The inventive device described herein additionally contains a programmable logic device (PLD) that is software configurable to efficiently allow additional processing capabilities, rectify/correct system deficiencies, and reconfiguration of selected input/output (I/O) pads.

In the system of this invention, a series of devices, e.g., pacemakers are provided which are based on a common hardware platform, and are flexibly software modifiable. By modifiable, it is meant that the control program can be changed, either upgrading or downgrading the program to increase or decrease the pacemaker sophistication and capability, allow correction of program anomalies, and/or allow evaluation of future features or algorithms in a clinical environment. Thus, a flexible common hardware platform, including microprocessor and associated memory, and conventional digital controller and timer circuitry, are provided.

### BRIEF DESCRIPTION OF THE DRAWINGS

FIG. 1 is a block diagram illustrating a programmable pacing system having an external programmer and an implantable pacemaker, with telecommunications capability between the programmer and the pacemaker;

FIG. 2 is a block diagram showing the primary components of an implantable pacemaker in accordance with this invention;

FIG. 3 is a block diagram showing additional aspects of an implantable pacemaker in accordance with this invention; and

FIG. 4 is a block diagram showing further details of an implantable pacemaker in accordance with this invention.

### DESCRIPTION OF THE PREFERRED EMBODIMENTS

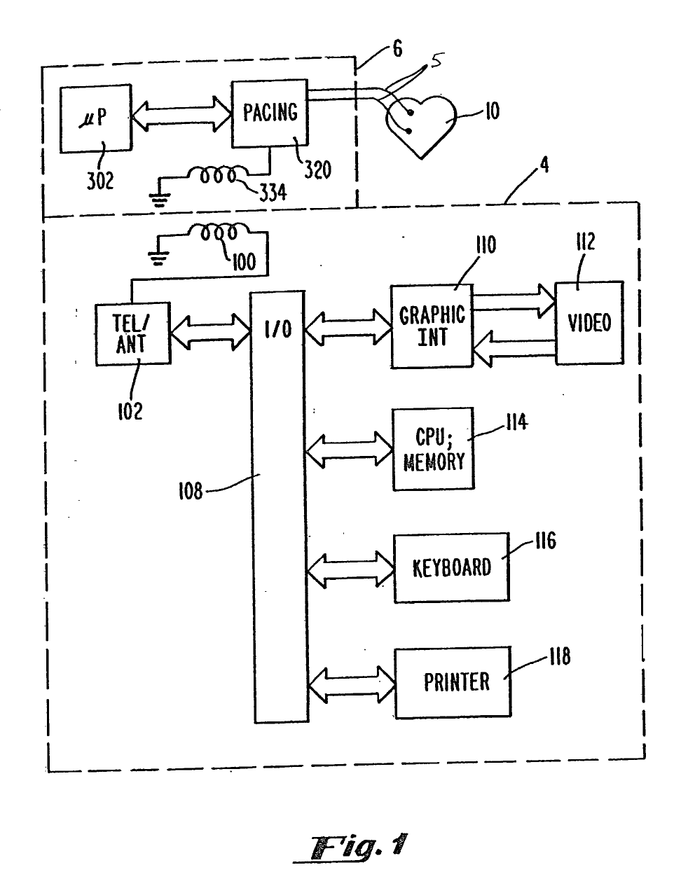

Referring now to FIG. 1, a pacemaker 6 is illustrated in block diagram form, coupled to a human heart 10 via leads 5. Also shown is an external programmer/display apparatus 4, of a type commercially available for programming multi-programmable implantable pacemakers. Within the housing of the pacemaker there is located pacing circuitry 320, which includes circuitry performing all of the basic timing, stimulation and sensing functions of a cardiac pacemaker, and a microprocessor circuit 302, which controls the timing intervals provided by the pacing circuitry 320 and performs other logic functions. Pacing circuitry 320 also includes a bi-directional telemetry circuit coupled to an antenna 334, allowing transmission of information (i.e., downlink) from external programmer 4 to pacemaker 6, and allowing transmission of information (i.e., uplink) from the pacemaker 6 to the programmer 4, corresponding to telemetry and programming systems presently available. The transmission of data from the programmer to the pacemaker may consist of modifying pacing parameters, or may constitute downloading of a new program to be stored with microprocessor 302, for controlling pacemaker functions. Data transmission from pacemaker 6 to programmer 4 may include data representative of the pacemaker, as set forth above, and may also include diagnostic data, which has been obtained and stored by the pacemaker.

Programmer 4, which is used by the physician, includes a corresponding antenna 100 for communicating with the pacemaker, the antenna being coupled to a telemetry/antenna driver circuit 102 which serves to demodulate telemetry signals received from antenna 334 of the pacemaker, and to apply them in parallel or serial digital format to input/output (I/O) unit 108, where they in turn may be applied to a video monitor 112 via graphic interface 110, and/or provided to central processing unit and memory 114, and/or printer 118. Unit 114 includes a microprocessor for controlling operation of the programmer/display apparatus, and is responsive to entered commands via keyboard 116, for controlling programming signals sent to the pacemaker, as well as for controlling operation of the video display 112 and printer 118. Unit 114 contains suitable memory for storing a plurality of software programs, e.g., control programs corresponding to different pacemaker types as discussed above.

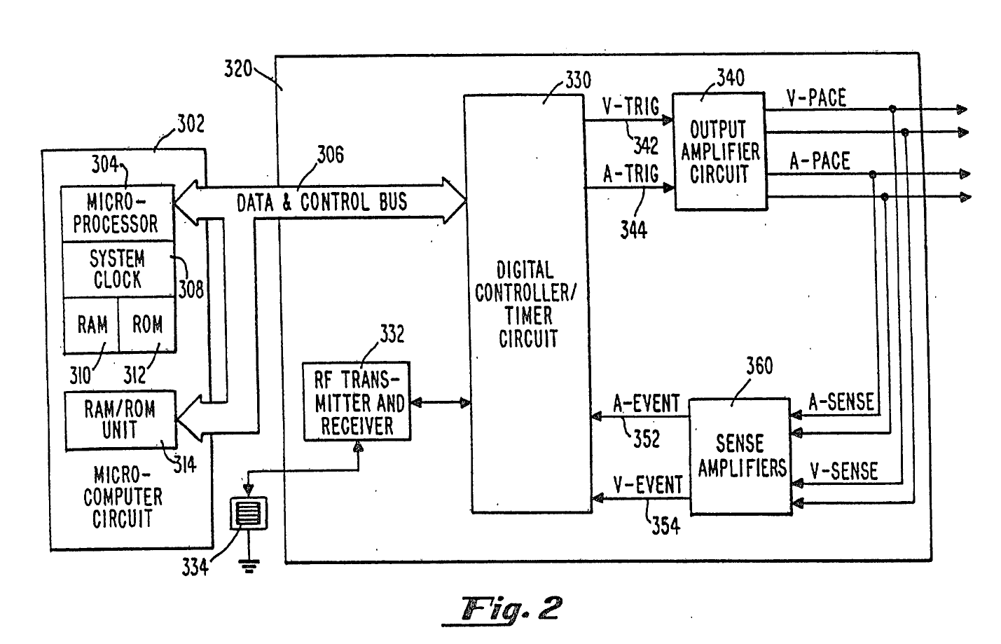

FIG. 2 is a block functional diagram of the pacemaker 6 illustrated in FIG. 1. The pacemaker is divided schematically into a microcomputer circuit 302 and a pacing circuit 320. The block diagram of FIG. 2 is representative of a dual chamber pacemaker, and accordingly pulse generator circuit 340 includes a ventricular pulse generator circuit coupled to the heart by a pair of V-pace output lines as well as an atrial pulse generator circuit coupled to the heart by means of atrial lines designated A-pace. Also represented at 360 are atrial and ventricular sense amplifiers. The output circuit 340 and sense amplifier circuits 360 may contain pulse generators and sense amplifiers corresponding to any of those presently employed in modern pacemakers. Control of timing and other functions within the pacemaker circuit is provided by digital controller/timer circuit 330, which includes a set of timers and associated logic. Digital controller/timer circuit 330 defines the pacing interval of the device, which may take the form of an A—A escape interval initiated on atrial sensing or pacing and triggering atrial pacing at the expiration thereof, or may take the form of a V—V interval initiated on ventricular sensing or pacing and triggering ventricular pulse pacing at the expiration thereof. Digital controller/timer circuit 330 similarly defines the A-V escape interval for a dual chamber pacemaker providing synchronous pacing. The specific values of the interval defined are controlled by the microcomputer circuit 302 by means of data and control bus 306. Sensed atrial depolarizations are communicated to the digital controller/timer circuit 330 on A-event line 352, ventricular depolarizations are communicated to digital control/timer circuit 330 on V-event line 354. In order to trigger generation of a ventricular pacing pulse, digital controller/timer circuit 330 generates a trigger signal on V trig line 342; similarly, in order to trigger an atrial pacing pulse, digital controller/timer circuit 330 generates a trigger pulse on A-trig line 344.

Transmission to and from the external programmer 4 is accomplished by means of antenna 344 and associated RF transmitter and receiver 322, which serves both to demodulate received downlink telemetry and to transmit uplink telemetry, all in a well-known manner. Microcomputer circuit 302 controls the operational functions of digital controller/timer 330, specifying which timing intervals are employed, and controlling the duration of the various timing intervals, via data and control bus 306. Microcomputer circuit 302 contains a base microprocessor 304, associated system clock 308, RAM 310, ROM 312. In addition, circuit 302 may include a separate RAM/ROM chip 314.

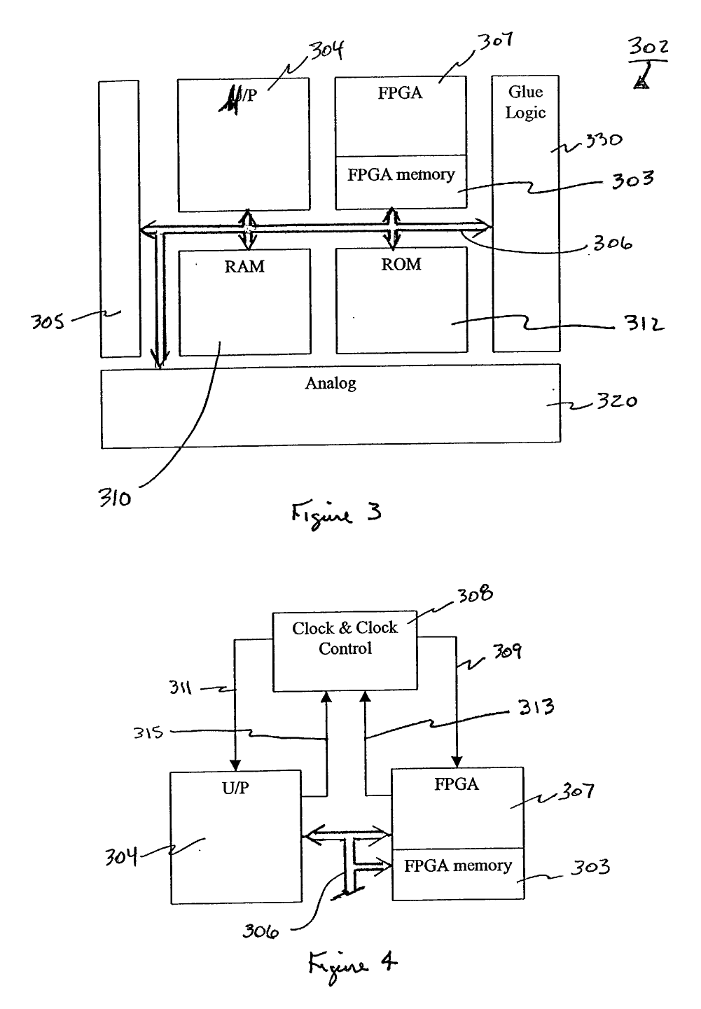

Referring now to FIG. 3, there is shown a more detail implementation of the microcomputer circuit 302 and I/O and glue logic circuit 320 of the present invention. Base microprocessor 304, RAM 310, ROM 312, glue logic 330, Input/Output (I/O) 305, PLD block 307, and analog circuit 320 are show interconnected by bus 306. The PLD block 307 may be programmed (i.e., configured) by data downloaded from production test equipment utilized during the manufacturing process or, alternatively, from an external programmer to an implanted device. The PLD memory block 303 may consist of either SRAM, EEPROM, flash or FRAM based memory. This memory holds the data that configures the PLD into a specific configuration. Alternatively, the memory 303 and thus the PLD 307 may be configured dynamically under control of the base processor 304. This allows dynamic alterable function by the base processor 304 in an implanted device 6.

A flexible microcomputer circuit 302 configured in accordance with the present invention can be implemented using any type of PLD array 307, many of which are well known in the art, such as a field programmable gate array (FPGA), a system programmable gate array (SPGA), a programmable array logic circuit (PAL), a programmable logic array circuit (PLA) or equivalent. Exemplary PLDs are described in U.S. Pat. No. 6,038,386 to Jain and U.S. Pat. No. 6,411,124 to Lee et al, both incorporated herein by reference in their entireties.

The PLD 307 configurable block may be used to implement a coprocessor to increase or enhance pacemaker 6 features and functions by increasing the system processing capabilities and speed. PLD 307 configurable block may be used to implement additional random “glue” logic may be used to increase or enhance pacemaker 6 features/functions and/or to reduce battery current drain. PLD 307 configurable block may additionally be used to modify or enhance base processor 304 op codes to increase processing power and/or reduce battery current drain. Further, PLD 307 configurable block may be used to modify I/O pad configurations to allow extensibility of function and/or allow additional product family differentiation without the need for a new IC design or redesign, saving significant time to market and R&D expenses. Additionally, the flexibility and capabilities of the inventive system allow the use of the temporary download clinical studies, algorithm development, diagnostic data gathering (for research or clinical validation studies), and system self test capabilities (for production testing/evaluation and/or clinical in-vivo problem solving/monitoring).

Referring now to FIG. 4, there is shown a more detailed block diagram of the clock control system of the present invention. Base processor 304 and PLD 307 with PLD memory 303 are shown interconnected with bus 306 (only partially shown). Clock and clock control block 308 are connected to base processor 304 by clock control line 315 and processor clock 311. Clock and clock control block 308 are connected to PLD 307 by clock control line 313 and PLD clock 309. Clock control circuit 308, under control from processor 304 and PLD 307, provides clock signals to the processor 304 and PLD 307. Clock control circuit 308 may stop, slow and synchronized clocks to each block independently. This allows the processor 304 to be suspended or slowed when the PLD is processing data or an algorithm. Clock control circuit 308 may use a lookup table to count clocks “ticks” for complete PLD processing before restarting the processor 304 clock. Alternatively, an interrupt schema may be used whereby the processor 304 firmware may provide a jump command to continue processing in the PLD, slow it's clock input via clock control circuit 308, and waiting for a flag or interrupt from the completion of processing of the algorithm in the PLD 307. This technique allows for increased processing power, capabilities and speed, enhanced function and reduced battery current drain.

To minimize additional static leakage battery current drain of pacemaker 6, the power to the PLD 307 and PLD memory 303 may be turned off when no active program is downloaded into PLD memory 303. A FET CMOS switch (not shown) may disconnect the power to PLD 307 and PLD memory 303.

The control of the revision levels and downloadable code programs stored in PLD memory 303 may be as described in the above referenced U.S. Pat. No. 5,843,138.

From the foregoing detailed descriptions of a particular embodiment of the invention, it should be apparent that an implantable medical device has been disclosed which provides a flexible platform allowing non-invasive redefinition of the pacing function. Incorporation of a PLD allows the device function to be re-defined while at the same time ensuring that battery power is reduced to a low level.

While a particular embodiment of the present invention has been described herein in detail, it is to be understood that various alterations, modifications, and substitutions can be made therein without departing from the spirit and scope of the present invention, as defined in the claims, which follow. In particular, it is contemplated by the inventor that the present invention may be incorporated into various different types of implanted, microprocessor-controlled medical devices, such as implantable pacemakers, cardioverters, defibrillators, neural stimulators, drug-administering devices, or other implantable devices, which automatically administer therapy to a patient under control of a predefined operational algorithm.

What is claimed is:

1. A programmable logic device (PLD) implanted in an implantable medical device (IMD) wherein a microcomputer circuit integrates the PLD with a base processor of the IMD, the microcomputer circuit comprising:
a PLD connected to a bus in the base processor;
a plurality of system clocks; and
a memory bank including I/O;
said PLD being operable to store downloaded software from an external programmer device to thereby reprogram the IMD in cooperation with said plurality of system clocks and said memory bank including I/O.

2. The circuit of claim 1 wherein said PLD further comprises:
a configurable block; and
a memory block.

3. The circuit of claim 2 wherein the memory block includes one of an SRAM, EEPROM and flash or FRAM based memory.

4. The circuit of claim 2 wherein the configurable block includes one of the type of field programmable gate array (FPGA), a system programmable gate array (SPGA), a programmable array logic circuit (PAL) and a programmable logic array circuit (PLA).

5. The circuit of claim 2 wherein said configurable block includes means for increasing system processing and speed of the base processor.

6. The circuit of claim 2 wherein said configurable block includes random glue logic to increase functionality of the base processor and reduce battery current drain.

7. The circuit of claim 2 wherein said configurable block includes means for increasing processor power of the base processor and reduce battery current drain.

8. The circuit of claim 7 wherein said means for increasing processor power includes means for modifying the base processor OP codes.

9. The circuit of claim 2 wherein said configurable block includes means for modifying the I/O to enable one of and combinations of extensibility of function and additional product family.

10. The circuit of claim 1 wherein temporary download of clinical studies, algorithmic development, diagnostic data gathering and system self-test are implemented in cooperation with said PLD, said plurality of system clocks and said memory block including I/O.

11. The circuit of claim 1 wherein said plurality of clocks control the base processor and the PLD.

12. The circuit of claim 1 wherein said clocks control one of and a combination of function to slow, stop and synchronize the base processor and the PLD.

13. The circuit of claim 11 wherein said plurality of clocks operates to turn power off to the PLD when not in use.
